(19) United States
(12) Patent Application Publication
(10) Pub. No.: US 2026/0309527 A1
(43) Pub. Date: Oct. 8, 2026

(54) METHOD FOR DETERMINING A FLOW RATE OF A FLUID INDEPENDENT OF THE THERMAL PROPERTIES OF THE FLUID

(71) Applicant: Berkin B.V., Ruurlo (NL)

(72) Inventors: Shirin Azadi KENARI, Ruurlo (NL); Joost Conrad LÖTTERS, Ruurlo (NL); Remco Gerardus Petrus SANDERS, Ruurlo (NL); Remco John WIEGERINK, Ruurlo (NL)

(21) Appl. No.: 19/144,879

(22) PCT Filed: Jan. 2, 2024
(86) PCT No.: PCT/NL2024/050002

§ 371 Date: Jun. 30, 2025

(30) Foreign Application Priority Data
Jan. 2, 2023 (NL) 2033895

(51) Int. Cl. G01F 1/696; A61M 16/00; G01F 1/684; G01F 1/69; G01N 25/18

(52) U.S. Cl. CPC G01F 1/696; A61M 16/0003; G01F 1/6845; G01F 1/69; G01N 25/18; A61M 2016/0027; A61M 2016/003; A61M 2205/3334

(57) ABSTRACT

The invention relates to a method for determining a flow rate of a fluid (1) independent of the physical properties of the fluid comprising:
placing a thermal flow sensor (2) in thermal contact with a fluid flow (3);
measuring flow rate;
placing a thermal property sensor (4) in a measurement cavity (5) in fluidic contact with the fluid flow, wherein the measurement cavity is fully open to the fluid flow;
receiving a portion (6) of the fluid in the measurement cavity of the thermal property sensor, in such a way, that the portion of the fluid is essentially stationary in the measurement cavity;
measuring at least one thermal property (κ, ρcp) of the fluid; and
compensating the measured flow rate for the at least one measured thermal property.

### FIELD OF THE INVENTION

The present invention generally relates to a method for determining a flow rate of a fluid independent of the thermal properties of the fluid, a method for determining a thermal conductivity (κ) and a heat capacity (cₚ) of a fluid, whose flow rate is to be determined, a thermal property sensor for use with a thermal flow sensor, wherein fluid, of which a flow rate is to be determined, flows through the thermal flow sensor during use, a thermal flow sensor for measuring a fluid flow, as well as a method for producing a thermal property sensor.

### BACKGROUND OF THE INVENTION

Thermal flow sensors are used to measure the flow rate of both gases and liquids. There are mainly three types of thermal flow sensors: anemometric, calorimetric and time-of-flight. Today, these sensors can be microdevices.

All three mentioned thermal flow sensor types are typically composed of a heater and temperature sensors; and follow a similar working principle, i.e., supplying power to the heater to elevate the temperature, and then measuring the change in temperature distribution over the sensor structure as a measure for the flow rate. Variations are possible, such as where power is kept constant and heaters also serving as temperature sensors.

Thermal flow sensors have a simple working principle and relatively low fabrication cost. However, they are dependent on the type of the flowing medium, more specifically the thermal properties of the gas or liquid. It means the fluid and its properties must be known for an accurate flow measurement.

In “Modelling and simulation of a thermal flow sensor for determining the flow speed and thermal properties of binary gas mixtures”, by Y. Q. Zhu, EUROSENSORS 2016, pp. 1028-1031, a thermal flow sensor was used consisting of a heater and a downstream temperature sensor to measure the thermal conductivity of the gas. The temperature of the downstream temperature sensor in a specific flow region is only dependent on κ.

In “Multi-parameter monitoring of binary gas mixtures: concentration and flow rate by DC excitation of thermal sensor arrays””, by Christoph J. Hepp et al, in Sensors and Actuators A, p. 32-39, 2017, a thermal sensor capable of simultaneously determining gas concentrations of a binary gas mixture under flow conditions and its flow rate is disclosed. Heat capacity is determined in a flow range within which the flow sensor is not sensitive to flow rate, which limits the employability of the sensor (i.e. the sensor first needs to be brought to a relatively ”high flow range).

In “Measurement and simulation of the frequency response of a thermal flow sensor at different flow speeds”, by D. F. Reyes, in Sensors and Actuators A, 203, pp. 225-233, 2013, AC excitation was used to measure the voltage of a sensor close to the heater. They used the fact that by increasing the frequency, the thermal boundary layer will decrease and can be brought down close to the wall Therefore, due to the non-slip condition on the wall, the thermal exchange between the heater and sensor is independent of the velocity, so the heat transfer is only affected by physical properties of the gas or fluid, and not by the flow.

U.S. Pat. No. 4,685,331 B1 discloses a thermal mass flowmeter and controller, having a series of flow channels and blocked channels, which are micro-etched from a silicon substrate leaving an integral membrane suspended across the channels. The thermal mass flowmeter's sensor comprises a stagnant gas channel that extends parallel to an etched flow channel, also described as “blocked channel”. The heat transfer equation described in U.S. Pat. No. 4,685,331 B1 is not dependent upon sensor flow rate because the measurement is made in the blocked stagnant channel. Thus, if the fluid in the flow channel changes in composition, it will take a considerable time to diffuse into the blocked channel. In addition, different fluids may have different diffusion rates into the blocked channel. The field requires immediate response to changes in gas (mixture) composition and therefore strongly prefers in-line sensors.

Therefore, measuring a fluid-independent flow rate, in particular a gas-independent flow rate, remains a major challenge.

### OBJECT OF THE INVENTION

It is therefore an object of the invention to provide a method allowing for measuring a fluid-independent flow rate, in particular a gas-independent flow rate.

### SUMMARY OF THE INVENTION

The invention provides a method for determining a flow rate of a fluid independent of the thermal properties of the fluid comprising:
placing a thermal flow sensor in thermal contact with a fluid flow;
measuring a flow rate;
placing a thermal property sensor in a measurement cavity in fluid connection with, and preferably adjacent to, the fluid flow;
receiving a portion of the fluid in the measurement cavity of the thermal property sensor, in such a way, that the portion of the fluid is essentially stationary in the measurement cavity, wherein the measurement cavity is fully open to the fluid flow;
measuring at least one thermal property (κ, ρcp) of the fluid, or a combination of thermal properties (κ, ρcp) of the fluid; and
compensating the measured flow rate for the at least one measured thermal property.

The above method basically advantageously calls for implementing the thermal property sensor in a “dead volume”, allowing for the measurement of at least one thermal property (κ, ρcₚ) of the fluid or a combination of thermal properties (κ, ρcp). The cavity is intentionally fully open to the flow. By knowing such thermal properties from the measurement, a fluid-independent flow rate measurement can be established.

The above-described technique is particularly suitable for thermal flow sensors inside channels, especially microelectromechanical thermal flow sensors in micromachined channels and further “fluidic chip” or “lab-on-a-chip” applications. However, the technique may also be used with heater and/or sensor elements e.g. fixed to an outer wall of a flow channel, wherein thermal contact with the fluid is established “indirectly”. Depending on the sensor type, the thermal flow sensor can be in thermal contact with the fluid flow directly or indirectly; for example hot wire sensors would be in direct contact, whereas the windings of capillary tube sensors would be in indirect contact.

In the context of the present patent application, “essentially stationary” means that the fluid basically “stands still”, but a relatively small degree of diffusion and movement of fluid particles in the measurement cavity is allowable, and even necessary to reflect changing fluid composition. In the present invention, it is found this is possible without interfering with the accuracy of the measurement. Grooves, especially V-grooves and U-grooves, are found to be particularly suitable for this purpose. They can be most advantageously used when facing the flow, or facing away from the flow.

An embodiment relates to an aforementioned method, comprising:
using the thermal flow sensor in the fluid flow to additionally measure at least one thermal property.

An embodiment relates to the addition of a second thermal flow sensor in the fluid flow.

Preferably, the thermal property measured using the thermal flow sensor is heat capacity (cₚ) or density (ρ) or volumetric heat capacity (ρcₚ).

An embodiment relates to an aforementioned method, wherein the thermal property measured on the essentially stationary fluid is thermal conductivity (κ).

A further embodiment relates to an aforementioned method, wherein a combination of thermal properties is measured concurrently in the fluid flow and on the essentially stationary fluid, wherein the thermal property measured in flow is heat capacity (cₚ) or density (ρ) or volumetric heat capacity (pcₚ) and the thermal property measured on the essentially stationary fluid is thermal conductivity (κ). Measurement of the volumetric heat capacity (ρcp) is also possible on the essentially stationary fluid. In addition, (κ/ρcₚ) may be determined.

An embodiment relates to an aforementioned method, wherein a pressure sensor additionally measures pressure and/or a pressure differential to derive viscosity from the measured flow rate and the measured pressure and/or the pressure differential. The skilled person would, for example, consult Chapter 2 of “Distributed thermal micro sensors for fluid flow”, by John van Baar, 2002 ISBN 90-36518288, and/or “Micro-machined structures for thermal measurements of fluid and flow parameters”, by J. J. van Baar, et al, in Journal of Micromechanics and Microengineering, volume 11, issue 4 (July), pp. 311-318, 2001. The latter article, like U.S. Pat. No. 4,685,331 B1, discloses a sensor in a “dead volume” which would inevitably contain stagnant fluids. In this set-up too, a change in fluid in the channel will take considerable time to diffuse into the blocked channel. In addition, different fluids may have different diffusion rates into the blocked channel. Van Baar (2001) also discloses a relatively large thermal conductivity sensor of 1 mm long by 25 μm wide by 1 μm thick as well as a TCR (“thermal coefficient of resistance”) sensor that is a thick silicon beam 1 mm long by 150 μm wide by 100 μm thick. The larger the volume, the larger the thermal capacitance of the structure, therefore, the slower the response of the sensor.

An embodiment relates to an aforementioned method, wherein the thermal property sensor comprises a heating wire, such as a pair of heating wires, or a probe or windings. Such a heating wire is relatively thin (compared to the volume of the measurement cavity) and therefore as such does not significantly influence the thermal properties of the essentially stationary fluid there. Optionally the thermal property sensor comprises two or more heating wires, which can serve as back-up for each other or check for drift. A particular advantage of wires is their fast response, typically <1 ms, which the current invention uses to increase speed of measurement. The skilled person will understand that instead of a heating wire other elements with a low thermal mass can be used.

Another embodiment relates to an aforementioned method, wherein the thermal property sensor comprises a probe, preferably having a sensor element made of platinum.

An embodiment relates to an aforementioned method, wherein thermal conductivity (κ) is measured by:
heating the heating wire with a constant current (DC) or very low frequency alternating current (AC), for heating the portion of the fluid, and
measuring a voltage of the heating wire during the heating of the portion of the fluid with voltage measurement means connected to the heating wire and relating the measured voltage to a thermal conductivity.

An embodiment relates to an aforementioned method, wherein heat capacity (cₚ) is measured by:
activating the heating wire with an alternating current (AC); and
measuring a phase and amplitude of the third harmonic (3ω) of the measured alternating current (AC) voltage of the heating wire during the heating of the portion of the fluid with voltage measurement means connected to the heating wire and relating the measured phase and amplitude of the third harmonic of the measured AC voltage to a heat capacity.

AC excitation can thus be advantageously used to measure the voltage of the thermal property sensor. The insight is used that by increasing the frequency, a thermal boundary layer thickness will decrease and can be brought down close to the wall. Therefore, due to the non-slip condition on the wall, the thermal exchange between a heater and sensor is independent of the velocity, so the heat transfer is only affected by physical properties of the gas or fluid, and not by the flow. Two physical parameters, thermal conductivity κ and volumetric heat capacity ρcₚ, are derived from the phase and amplitude of the third harmonic of the measured AC voltage. The skilled person will take the type of sensor and other environmental factors into account when selecting a suitable frequency. The flow rate itself can be measured with DC excitation which is dependent on κ and ρcₚ. It will be clear to the skilled person that very low frequency AC excitation can be used as an alternative for DC excitation, if the frequency is so low that measurement can be completed in a fraction of a period, such as half a period or less. The skilled person will of course take into account the type of fluid, and any additional measures to compensate. Therefore, by knowing these properties from the AC measurement, a fluid/gas independent flow rate measurement can be achieved.

Another aspect of the invention relates to a method for determining a thermal conductivity (κ) and/or a heat capacity (cₚ) of a fluid, whose flow is to be determined, comprising:
placing a thermal flow sensor in thermal contact with a fluid flow;
measuring flow rate;
placing a thermal property sensor in a measurement cavity in fluid connection with, and preferably adjacent to, the fluid flow, wherein the measurement cavity is fully open to the fluid flow, such as facing (away from) the flow, e.g. being arranged in (the flow path of) the fluid flow;
receiving the portion of the fluid in the measurement cavity of the thermal property sensor, in such a way, that the portion of the fluid is essentially stationary in the measurement cavity,
heating a heating wire of the thermal property sensor with a constant current (DC) or very low frequency alternating current (AC), for heating the portion of the fluid, and
measuring the voltage of the heating wire during the heating of the portion of the fluid with voltage measurement means connected to the heating wire and relating the measured voltage to a thermal conductivity; and/or
heating the heating wire of the thermal property sensor with an alternating current (AC); and
measuring a phase and amplitude of the third harmonic of the measured alternating current (AC) voltage of the heating wire during the heating of the portion of the fluid with voltage measurement means connected to the heating wire and relating the measured phase and amplitude of the third harmonic of the measured AC voltage to a heat capacity (preferably volumetric heat capacity, ρcp).

In an embodiment, placing a thermal flow sensor in thermal contact with a fluid flow comprises thermal contact through an outer wall of a flow channel.

Another aspect of the invention relates to a thermal property sensor for use with a thermal flow sensor, wherein fluid, of which a flow rate is to be determined, flows through the thermal flow sensor during use, comprising:
a heating wire configured for being placed in a measurement cavity for receiving a portion of the fluid, in such a way, that the portion of the fluid is essentially stationary in the measurement cavity, the heating wire further being configured for being heated with:
a constant current (DC) or very low frequency alternating current (AC), for heating the portion of the fluid, wherein the voltage of the heating wire during the heating of the portion of the fluid is measured with voltage measurement means connected to the heating wire, wherein the measured voltage is related to a thermal conductivity; and/or
an alternating current (AC), wherein a phase and amplitude of the third harmonic of the alternating current (AC) voltage of the heating wire during the heating of the portion of the fluid are measured with voltage measurement means connected to the heating wire, wherein the measured phase and amplitude of the third harmonic of the measured AC voltage are related to a heat capacity (preferably volumetric heat capacity, ρcp).

An embodiment relates to an aforementioned thermal property sensor, wherein the measurement cavity is a groove and has a U-shaped or V-shaped cross-section, wherein the heating wire is suspended in the measurement cavity with the U- or V-shaped cross-section. This embodiment is particularly suitable to keep the fluid “essentially stationary” while permitting the diffusion necessary for fluid exchange.

An embodiment relates to an aforementioned thermal property sensor, wherein the wire has a cross-section of under 10 μm, such as about 9 μm, about 8 μm, 7 μm, about 6 μm, 5 μm, about 4 μm, 3 μm, about 2 μm, or about 1 μm, in order to achieve a small volume, and therefore a small thermal capacity and consequently a fast response.

An embodiment relates to an aforementioned thermal property sensor, wherein the wire has a cross-section that is flattened on one side, such as a square shape, a triangular shape, a semi-circular shape, or, most preferably a rectangular shape. The flat part of the flattened cross-section preferably has a width of under 10 μm, such as about 9 μm, about 8 μm, 7 μm, about 6 μm, 5 μm, about 4 μm, 3 μm, about 2 μm, or about 1 μm. In addition the cross-section preferably has a thickness of 0.1-1 μm, depending on the robustness of the material and the conditions. The small volume of the sensor allows for extremely high speed. This has been estimated for silicon, where the time constant scales linearly with the volume, thus reducing the volume by half will increase the speed of a sensor about 2 times if all other variables remain the same. A response that is twice as fast would be desirable in the field of flow sensors, and even larger increases are possible by reducing the volume by more than half.

An embodiment relates to an aforementioned thermal property sensor, wherein the measurement cavity with the U- or V-shaped cross-section and/or the heating wire has a length of 1-3 mm, such as 1.5-2.5 mm, and/or the measurement cavity with the U- or V-shaped cross-section has a width of 20-60 μm.

An embodiment relates to an aforementioned thermal property sensor, wherein the thermal property sensor is placed to minimize distance to the thermal flow sensor, i.e. without taking the thermal property sensor out of the cavity.

Another aspect of the invention concerns a thermal flow sensor for measuring a fluid flow, comprising:
a measurement cavity in fluid connection with, and preferably adjacent to, the fluid flow, wherein the measurement cavity is fully open to the fluid flow;
an aforementioned thermal property sensor, wherein the heating wire is placed in the measurement cavity for receiving a portion of the fluid, in such a way, that the portion of the fluid is essentially stationary in the measurement cavity.

An embodiment relates to an aforementioned thermal flow sensor, wherein the thermal flow sensor is releasably inserted into a flow channel in which a fluid flow is present during use or, when thermal contact occurs (indirectly) through an outer wall of a flow channel, fixed to an outer wall of a flow channel.

An embodiment relates to an aforementioned thermal flow meter or controller, comprising the aforementioned thermal property sensor and/or the thermal flow sensor.

An embodiment relates to an aforementioned thermal flow meter or controller, wherein at least one of the thermal property sensor or the thermal flow sensor is a microelectromechanical (MEMS) device.

An embodiment relates to an aforementioned thermal flow meter or controller, further comprising a viscosity sensor, (external) humidity sensor, CO₂ sensor, (external) temperature sensor, dielectric or permittivity sensor, additional pressure sensor, fluid composition sensor or multiparameter sensor.

Another aspect of the invention concerns a use of the aforementioned thermal flow meter or controller in a medical device.

The use may be in a respiratory device.

Another aspect of the invention concerns a method for producing an aforementioned thermal sensor flow sensor, comprising the steps of:
(1) depositing a support layer at both sides of a wafer,
(2) depositing a metal layer on one side of the wafer,
(3) patterning the metal layer,
(4) patterning the support layer to open windows for etching the Si underneath,
(5) repeating steps 2 to 4 on the other side of the wafer,
(6) etching the Si wafer to realize a V-groove and a flow sensing element cavity inside the wafer.

An embodiment relates to an aforementioned production method, wherein:
(4) the support layer is etched to open the window for etching the Si wafer underneath.

An embodiment relates to an aforementioned production method, wherein the metal layer comprises a Cr/Pt layer.

An embodiment relates to an aforementioned production method, wherein the support layer is an SiRN support layer.

An embodiment relates to an aforementioned method, wherein:
(1) the support layer has a thickness of 1 μm.

An embodiment relates to an aforementioned method, wherein:
(2) the layer of Cr/Pt has a thickness of 20 nm/ 200 nm,

An embodiment relates to an aforementioned method, wherein:
(3) patterning the support layer comprises etching of the Cr/Pt layer,

An embodiment relates to an aforementioned method, wherein:
(2) the layer of Cr/Pt is deposited by sputtering.

In an embodiment, Step 1 uses Low Pressure Chemical Vapor deposition (LPCVD).

In an embodiment, Step 6 uses KOH for the etching, preferably 1:3 in distilled water.

An embodiment relates to an aforementioned thermal flow sensor, comprising:
a sensor body with a flow section, through which the fluid, of which a flow rate is to be determined, flows in a flow direction during use,
a flow sensor configuration, comprising one or more flow sensing elements arranged at one or more locations in the flow section.

An embodiment relates to an aforementioned thermal flow sensor, comprising:
a main body portion;
a first body portion extending from the main body portion;
a second body portion extending from the main body portion;
wherein the flow section is formed between the main body portion, the first body portion and the second body portion.

An embodiment relates to an aforementioned thermal flow sensor, wherein the measurement cavity is arranged in the main body portion.

An embodiment relates to an aforementioned thermal flow sensor, wherein the measurement cavity extends along a side of the flow section.

An embodiment relates to an aforementioned thermal flow sensor, wherein the flow sensor configuration comprises multiple flow sensing elements arranged at multiple locations in the flow section.

An embodiment relates to an aforementioned thermal flow sensor, wherein the flow section is open at a side of the flow section not delimited by the main body portion, the first body portion and/or the second body portion.

An embodiment relates to an aforementioned thermal flow sensor, wherein the first body portion and the second body portion are parallel to each other.

An embodiment relates to an aforementioned thermal flow sensor, wherein the flow section has a square or rectangular shape in a plane transversal to the flow direction.

An embodiment relates to an aforementioned thermal flow sensor, wherein the multiple flow sensing elements extend between the first body portion and the second body portion.

An embodiment relates to an aforementioned thermal flow sensor, wherein the multiple flow sensing elements are spaced-apart in the flow section in an even manner.

An embodiment relates to an aforementioned thermal flow sensor, wherein the multiple flow sensing elements comprise three or more flow sensing elements.

An embodiment relates to an aforementioned thermal flow sensor, wherein each flow sensing element comprises a pair of flow sensing wires.

An embodiment relates to an aforementioned thermal flow sensor, wherein a mutual distance between the pairs of flow sensing wires is 300-500 μm, preferably 350-450 μm, more preferably 375-425 μm.

An embodiment relates to an aforementioned thermal flow sensor, wherein the pairs of flow sensing wires of the thermal flow sensor extend between the first body portion and the second body portion.

An embodiment relates to an aforementioned thermal flow sensor, wherein the sensor body is formed as a chip.

An embodiment relates to an aforementioned thermal flow sensor, wherein the sensor body is attached to, or arranged on, a printed circuit board (PCB).

An embodiment relates to an aforementioned thermal flow sensor, wherein at least one of the sensors, preferably being a flow sensing wire, form part of a Wheatstone bridge.

An embodiment relates to an aforementioned thermal flow sensor, wherein each of the one or more pairs of the sensors, preferably being flow sensing wires, forms one half of a Wheatstone bridge.

An embodiment relates to an aforementioned thermal flow sensor, wherein fixed resistors arranged on the sensor body form the other half of the Wheatstone bridge.

An embodiment relates to an aforementioned thermal flow sensor, wherein the thermal flow sensor is a microelectromechanical system (MEMS) component.

### BRIEF DESCRIPTION OF THE DRAWINGS

The invention will be explained by means of the exemplary embodiments depicted in the accompanying drawings and the detailed description of the Figures below.

FIG. 1 shows a perspective view of an example embodiment of a thermal flow sensor;

FIG. 2 shows a perspective view of an example embodiment of a thermal property sensor with a measurement cavity;

FIG. 3 shows an example embodiment of a heating wire of a thermal property sensor connected to voltage measurement means;

FIG. 4 shows a cross-section of an example embodiment of a heating wire arranged in a measurement cavity with a V-shaped cross-section;

FIG. 5 shows a circuit schematic of a Wheatstone bridge;

FIG. 6 shows fabrication steps for fabricating a V-shaped measurement cavity with a heating wire arranged therein; and

FIG. 7 shows another example embodiment of a flow channel with a thermal flow sensor and a thermal property sensor.

### DETAILED DESCRIPTION

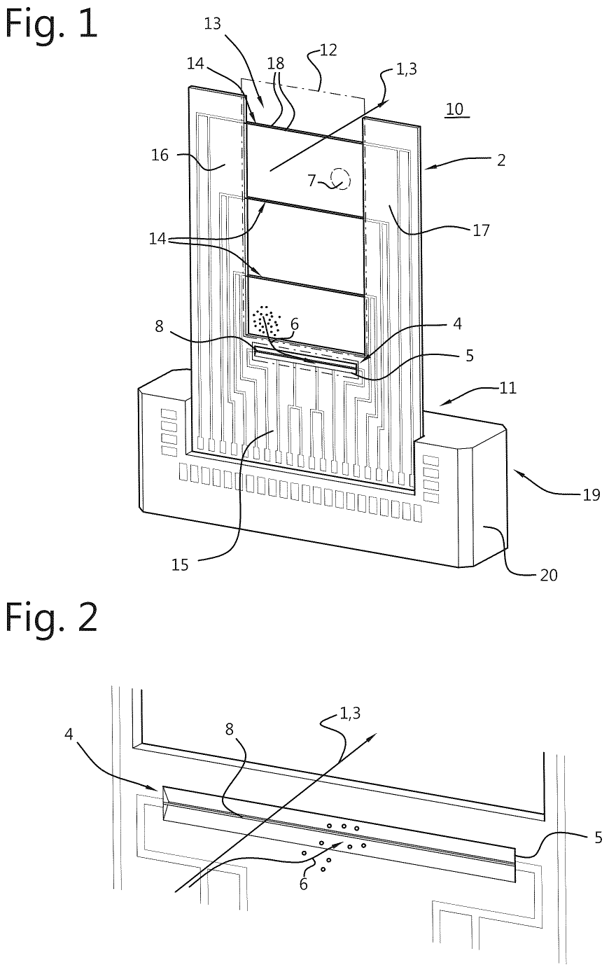

FIG. 1 shows a perspective view of an example embodiment of a thermal flow sensor 2 for measuring a fluid flow 3, comprising a measurement cavity 5 in fluid connection with, and preferably adjacent to, the fluid flow 3. The measurement cavity 5 is furthermore fully open to the fluid flow 3. The measurement cavity 5 may be arranged in the fluid flow 3, e.g. being immersed in the fluid flow 3. The measurement cavity 5 may face (away from) the fluid flow 3. The measurement cavity 5 is comprised by a thermal property sensor 4. A heating wire 8 is placed in the measurement cavity 5 for receiving a portion 6 of the fluid, in such a way, that the portion 6 of the fluid is essentially stationary in the measurement cavity 5. The thermal flow sensor 2 may be releasably inserted into a flow channel 10 in which a fluid flow 3 is present during use. FIG. 1 shows an embodiment of a chip with bond pads. However, the skilled person will understand that other designs or embodiments are also conceivable.

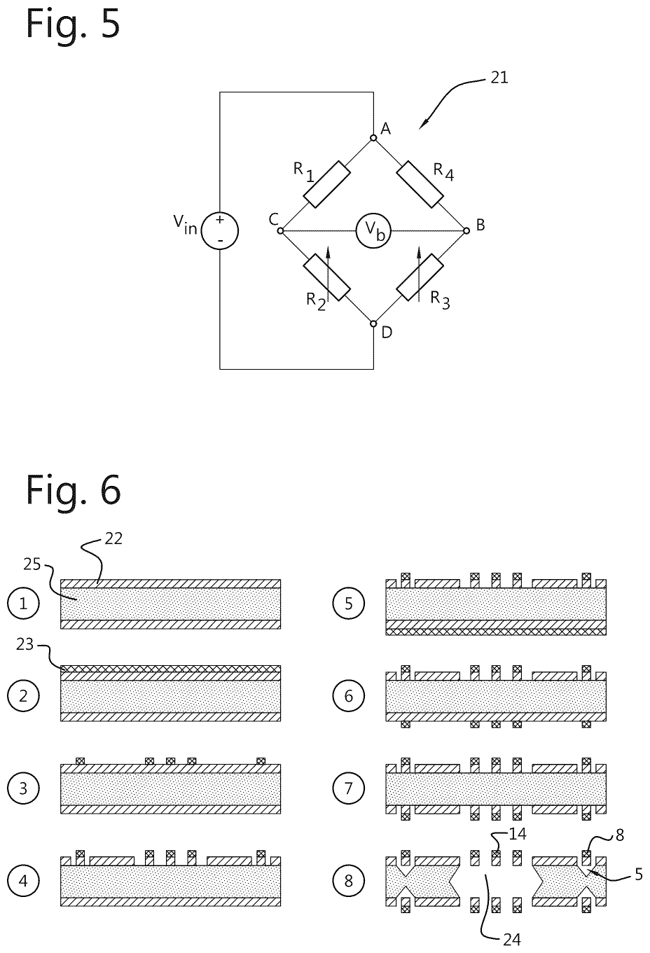

The thermal flow sensor 2 may comprise a sensor body 11 with a flow section 12, through which the fluid 1, of which a flow rate is to be determined, flows in a flow direction during use. A flow sensor configuration 13 may be provided, comprising one or more flow sensing elements 14 arranged at one or more locations in the flow section 12. The thermal flow sensor 2 may comprise a main body portion 15, a first body portion 16 extending from the main body portion 15 and a second body portion 17 extending from the main body portion 15. The flow section 12 may be formed between the main body portion 15, the first body portion 16 and the second body portion 17. The measurement cavity 5 may be arranged in the main body portion 15. The measurement cavity 5 may extend along a side of the flow section 12. The flow sensor configuration 13 may comprise multiple flow sensing elements 14 arranged at multiple locations in the flow section 12, such as three, as shown in FIG. 1. The flow section 12 may be open at a side of the flow section 12 not delimited by the main body portion 15, the first body portion 16 and/or the second body portion 17. The first body portion 16 and the second body portion 17 may be parallel to each other. The flow section 12 may have a square or rectangular shape in a plane transversal to the flow 3 direction. The multiple flow sensing elements 14 may extend between the first body portion 16 and the second body portion 17. The multiple flow sensing elements 14 can be spaced-apart in the flow section 12 in an even manner. Each flow sensing element 14 may comprise a pair of flow sensing wires 18. A mutual distance between the pairs of flow sensing wires 14 may be 300-500 μm, preferably 350-450 μm, more preferably 375-425 μm. The pairs of flow sensing wires 18 of the thermal flow sensor 2 may extend between the first body portion 16 and the second body portion 17. Such wires 18 were found to be surprisingly robust and insensitive to small particles, such as dust, that occasionally contaminate fluids. However, appropriate measures against overheating should be taken. The sensor body 11 may be formed as a chip 19. The sensor body 11 may be attached to, or arranged on, a printed circuit board (PCB) 20. Each of the one or more pairs of flow sensing wires 18 may form one half (R₂, R₃) of a Wheatstone bridge 21, as shown in FIG. 5. The skilled person will understand that the arrow shown through R₃ should point downwards in case a differential flow signal is to be measured. Fixed resistors R₁, R₄ arranged on the sensor body 11 may form the other half of the Wheatstone bridge 21. The thermal flow sensor 2 may be a microelectromechanical system (MEMS) component. In no-flow condition, R₁ and R₄ and R₂ and R₃ have the same value, so the output signal of the Wheatstone bridge 21 is zero. When flow is applied, heat will be transferred from an upstream wire to a downstream one. Therefore, there will be a positive or negative (depending on the flow direction) output voltage signal as a result of the temperature difference between the two wires R₂ and R₃.

One or more flow sensing elements 14 may also be arranged at (the outside of) an outer wall (not shown) of the schematically indicated flow channel 10, as to establish “indirect” thermal contact with the fluid flow 3.

FIG. 2 shows a perspective view of an example embodiment of a thermal property sensor 4 with a measurement cavity 5 in more detail.

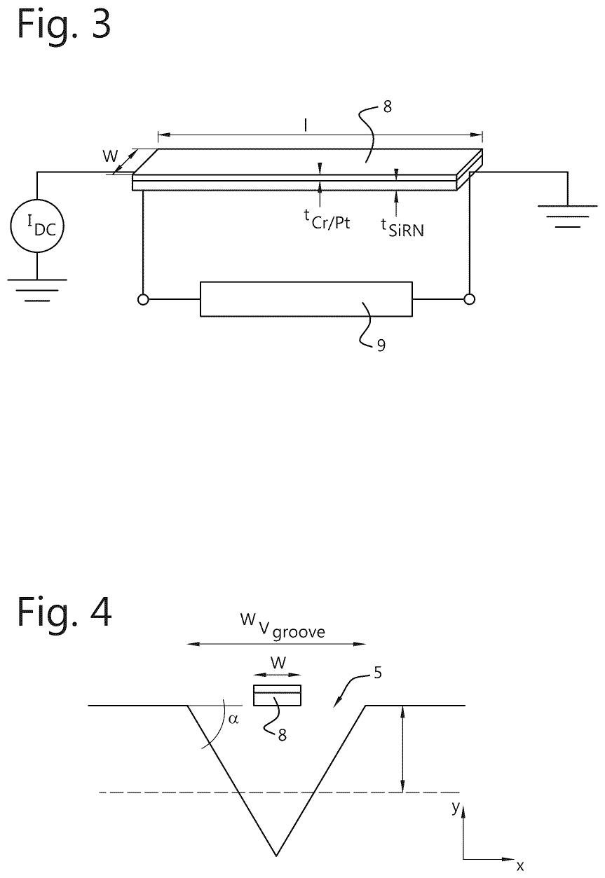

As more clearly shown in FIG. 3, the heating wire 8 may be configured for being heated with a constant current (DC) or very low frequency alternating current (AC), for heating the portion 6 of the fluid 1, wherein the voltage of the heating wire 8 during the heating of the portion 6 of the fluid is measured with voltage measurement means 9 connected to the heating wire 8, wherein the measured voltage is related to a thermal conductivity κ; and/or an alternating current (AC), wherein a phase and amplitude of the third harmonic of the alternating current (AC) voltage of the heating wire 8 during the heating of the portion 6 of the fluid 1 are measured with voltage measurement means 9 connected to the heating wire 8, wherein the measured phase and amplitude of the third harmonic of the measured AC voltage are related to a heat capacity cₚ.

As shown in FIG. 4, the measurement cavity 5 may have a V-shaped cross-section, wherein the heating wire 8 is suspended in the measurement cavity 5 with the V-shaped cross-section. The measurement cavity 5 with the V-shaped cross-section may have a length/of 1-3 mm and a width (W_{Vgroove}) of 20-60 μm in a MEMS embodiment. The temperature of the heating wire 8 is dominated by the thermal conductivity κ of the fluid/gas 6 inside the measurement cavity 5 and largely independent of the fluid flow 3 velocity. Hence, by monitoring the voltage drop over the heating wire 8 at constant heating current, κ can be detected. The angle α as shown in FIG. 4 may be 50-60 degrees.

According to the invention, a method for determining a flow rate of a fluid 1 independent of the thermal properties of the fluid 1 is provided, comprising:
contacting a thermal flow sensor 2 to a fluid flow 3 or generally placing a thermal flow sensor 2 in thermal contact with a fluid flow 3;
measuring flow rate;
placing a thermal property sensor 4 in a measurement cavity 5 in fluid connection with, and preferably adjacent to, the fluid flow 3, wherein the measurement cavity 5 is fully open to the fluid flow 3;
receiving a portion 6 of the fluid in the measurement cavity 5 of the thermal property sensor 4, in such a way, that the portion 6 of the fluid is essentially stationary in the measurement cavity 5;
measuring at least one thermal property (κ, ρcp) of the fluid 1; and
compensating the measured flow rate for the at least one measured thermal property.

The method may further comprise:
using the thermal flow sensor 2 in the fluid flow 3 to additionally measure at least one thermal property.

The thermal property measured in the fluid flow 3 may be heat capacity (cₚ) or density (ρ).

The thermal property measured on the essentially stationary fluid 6 is thermal conductivity (κ).

A highly schematically indicated pressure sensor 7 may additionally measure pressure to derive viscosity from the thermal property and the measured pressure (or measured pressure differential). Viscosity may also be measured “thermally”, as demonstrated by John van Baar.

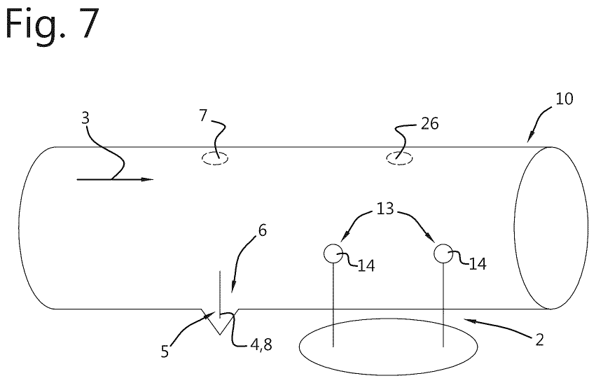

A highly schematically indicated additional sensor 26, such as shown in FIG. 7, may be added to the device. The additional sensor 26 may be a viscosity sensor, (external) humidity sensor, CO₂ sensor, (external) temperature sensor, dielectric or permittivity sensor, fluid composition sensor or multiparameter sensor.

The thermal conductivity (κ) can be measured by:
heating the heating wire 8 with a constant current (DC) or very low frequency alternating current (AC), for heating the portion 6 of the fluid 1, and
measuring a voltage of the heating wire 8 during the heating of the portion 6 of the fluid with voltage measurement means 9 connected to the heating wire 8 and relating the measured voltage to a thermal conductivity.

The heat capacity (cₚ) can be measured by:
activating the heating wire 8 with an alternating current (AC); and
measuring a phase and amplitude of the third harmonic of the measured alternating current (AC) voltage of the heating wire 8 during the heating of the portion 6 of the fluid with voltage measurement means 9 connected to the heating wire 8 and relating the measured phase and amplitude of the third harmonic of the measured AC voltage to a heat capacity (preferably volumetric heat capacity, ρcp).

In line with the invention, a method for determining a thermal conductivity (κ) and/or a heat capacity (cp) of a fluid 1, whose flow is to be determined, is provided, comprising:
contacting a thermal flow sensor 2 to a fluid flow 3;
measuring flow rate;
placing a thermal property sensor 4 in a measurement cavity 5 in fluid connection with, and preferably adjacent to, the fluid flow, wherein the measurement cavity 5 is fully open to the fluid flow, e.g. being arranged in the fluid flow;
receiving the portion 6 of the fluid in the measurement cavity 5 of the thermal property sensor 4, in such a way, that the portion 6 of the fluid is essentially stationary in the measurement cavity 5, and/or
heating a heating wire 8 of the thermal property sensor 4 with a constant current (DC) or very low frequency alternating current (AC), for heating the portion 6 of the fluid, and
measuring the voltage of the heating wire 8 during the heating of the portion 6 of the fluid with voltage measurement means 9 connected to the heating wire 8 and relating the measured voltage to a thermal conductivity;
heating the heating wire 8 of the thermal property sensor 4 with an alternating current (AC); and
measuring a phase and amplitude of the third harmonic of the measured alternating current (AC) voltage of the heating wire 8 during the heating of the portion 6 of the fluid with voltage measurement means 9 connected to the heating wire and relating the measured phase and amplitude of the third harmonic of the measured AC voltage to a heat capacity (preferably volumetric heat capacity, ρcp).

As shown in FIG. 6, the invention also relates to a method for producing an aforementioned thermal property sensor 4, wherein first, a support layer 22, preferably of 1 μm SiRN, is deposited on an Si wafer 25 by, for example, LPCVD (1). Then, a 20 nm Cr adhesion layer and 200 nm Pt layer 23 are deposited and etched by sputtering and IBE etching, respectively, to pattern the wires and metal traces (2, 3). The combination of Cr and Pt at these thicknesses gives excellent results, but other thicknesses and combinations metals are possible. The IBE etching step is performed twice with two different masks. The first step is for transferring the metal pattern, the second one to narrow the beam width and define the pattern in the SiRN support layer 22. In (4), SiRN is etched by plasma etching to open the window for etching the Si. All these steps are repeated for the backside of the wafer 25 to have wires 8, 14 on both sides (5-7). Finally, Si is etched by KOH (KOH 1:3 DI-water) to realize a cavity 5, 24 inside the wafer 25 between/around the wires 8, 14.

FIG. 7 shows an example embodiment of a flow channel 10 with a thermal flow sensor 2 and a thermal property sensor 4, wherein the flow sensing elements 14 may comprise probes. A pressure sensor 7 and an additional sensor 26 may be provided, to measure a differential pressure. The flow sensing elements 14 in the form of probes may be arranged at spaced-apart positions in the flow 3. The measurement cavity 5 with a heating wire 8 is also shown.

### LIST OF REFERENCE NUMERALS

1. Fluid
2. Thermal flow sensor
3. Fluid flow
4. Thermal property sensor
5. Measurement cavity
6. Stationary portion of the fluid
7. Pressure sensor
8. Heating wire
9. Voltage measurement means
10. Flow channel
11. Sensor body
12. Flow section
13. Flow sensing configuration
14. Flow sensing element
15. Main body portion
16. First body portion
17. Second body portion
18. Flow sensing wire
19. Chip
20. PCB
21. Wheatstone bridge
22. Support Layer (of SiRN)
23. Cr/Pt-layer
24. Flow sensing element cavity
25. Si wafer
26. Additional/further sensor

1-24. (canceled)

25. A method for determining a flow rate of a fluid independent of the physical properties of the fluid comprising:
placing a thermal flow sensor in thermal contact with a fluid flow;
measuring a flow rate;
placing a thermal property sensor in a measurement cavity in fluidic contact with the fluid flow;
receiving a portion of the fluid in the measurement cavity of the thermal property sensor, in such a way, that the portion of the fluid is essentially stationary in the measurement cavity, wherein the measurement cavity is fully open to the fluid flow;
measuring at least one thermal property (κ, ρcₚ) of the fluid, or a combination of thermal properties (κ, ρcp) of the fluid; and
compensating the measured flow rate for the at least one measured thermal property.

26. The method according to claim 25, comprising:
using the thermal flow sensor in the fluid flow to additionally measure at least one thermal property.

27. The method according to claim 26, wherein the thermal property measured using the thermal flow sensor is heat capacity (cₚ) or density (ρ) or volumetric heat capacity (ρcₚ).

28. The method according to claim 25, wherein the thermal property measured of the essentially stationary fluid is thermal conductivity (κ).

29. The method according to claim 25, wherein a pressure sensor additionally measures at least one of pressure and a pressure differential to derive viscosity from the measured flow rate and the at least one of the measured pressure and the pressure differential.

30. The method according to claim 25, wherein the thermal property sensor or the thermal flow sensor comprises a heating wire or a probe or windings.

31. The method according to claim 30, wherein thermal conductivity (κ) is measured by:
heating the heating wire with a constant current (DC) or very low frequency alternating current (AC), for heating the portion of the fluid, and
measuring a voltage of the heating wire during the heating of the portion of the fluid with voltage measurement means connected to the heating wire and relating the measured voltage to a thermal conductivity.

32. The method according to claim 30, wherein heat capacity (cₚ) is measured by:
activating the heating wire with an alternating current (AC); and
measuring a phase and amplitude of the third harmonic of the measured alternating current (AC) voltage of the heating wire during the heating of the portion of the fluid with voltage measurement means connected to the heating wire and relating the measured phase and amplitude of the third harmonic of the measured AC voltage to a heat capacity.

33. The method for determining a thermal conductivity (κ) and a heat capacity (cₚ) of a fluid, whose flow is to be determined, comprising:
placing a thermal flow sensor in thermal contact with a fluid flow;
measuring flow rate;
placing a thermal property sensor in a measurement cavity in fluid connection with the fluid flow, wherein the measurement cavity is fully open to the fluid flow;
receiving the portion of the fluid in the measurement cavity of the thermal property sensor, in such a way, that the portion of the fluid is essentially stationary in the measurement cavity,
heating a heating wire of the thermal property sensor with a constant current (DC) or very low frequency alternating current (AC), for heating the portion of the fluid, and
measuring the voltage of the heating wire during the heating of the portion of the fluid with voltage measurement means connected to the heating wire and relating the measured voltage to a thermal conductivity;
heating the heating wire of the thermal property sensor with an alternating current (AC); and
measuring a phase and amplitude of the third harmonic of the measured alternating current (AC) voltage of the heating wire during the heating of the portion of the fluid with voltage measurement means connected to the heating wire and relating the measured phase and amplitude of the third harmonic of the measured AC voltage to a heat capacity.

34. The method according to claim 25, wherein placing a thermal flow sensor in thermal contact with a fluid flow comprises thermal contact through an outer wall of a flow channel.

35. A thermal property sensor for use with a thermal flow sensor, wherein fluid, of which a flow rate is to be determined, flows through the thermal flow sensor during use, comprising:
a heating wire configured for being placed in a measurement cavity for receiving a portion of the fluid, in such a way, that the portion of the fluid is essentially stationary in the measurement cavity, the heating wire further being configured for being heated with at least one of:
a constant current (DC) or very low frequency alternating current (AC), for heating the portion of the fluid, wherein the voltage of the heating wire during the heating of the portion of the fluid is measured with voltage measurement means connected to the heating wire, wherein the measured voltage is related to a thermal conductivity; and
an alternating current (AC), wherein a phase and amplitude of the third harmonic of the alternating current (AC) voltage of the heating wire during the heating of the portion of the fluid are measured with voltage measurement means connected to the heating wire, wherein the measured phase and amplitude of the third harmonic of the measured AC voltage are related to a heat capacity.

36. The thermal property sensor according to claim 35, wherein the measurement cavity is a groove and has a U- or V-shaped cross-section, wherein the heating wire is suspended in the measurement cavity with the U- or V-shaped cross-section.

37. The thermal property sensor according to claim 36, wherein at least one of the measurement cavity with the V-shaped cross-section or the heating wire has a length of 1-3 mm and the measurement cavity with the V-shaped cross-section has a width (W_{Vgroove}) of 20-60 μm.

38. A thermal flow sensor for measuring a fluid flow, comprising:
a measurement cavity in fluid connection with the fluid flow, wherein the measurement cavity is fully open to the fluid flow;
a thermal property sensor according to claim 35, wherein the heating wire is placed in the measurement cavity for receiving a portion of the fluid, in such a way, that the portion of the fluid is essentially stationary in the measurement cavity.

39. The thermal flow sensor according to claim 38, wherein the thermal flow sensor is releasably inserted into a flow channel in which a fluid flow is present during use or fixed to an outer wall of a flow channel.

40. A thermal flow meter or controller, comprising the thermal property sensor of claim 35.

41. The thermal flow meter or controller according to claim 40, wherein at least one of the thermal property sensor or the thermal flow sensor is a microelectromechanical device.

42. The thermal flow meter or controller according to claim 40, further comprising a viscosity sensor, (external) humidity sensor, CO₂ sensor, (external) temperature sensor, dielectric or permittivity sensor, fluid composition sensor or multiparameter sensor.

43. A use of the thermal flow meter or controller according to claim 40 in a medical device.

44. A method for producing a thermal flow sensor according to claim 38, comprising the steps of:
(1) depositing a support layer at both sides of a wafer,
(2) depositing a metal layer on one side of the wafer,
(3) patterning the metal layer,
(4) patterning the support layer to open windows for etching the Si underneath,
(5) repeating steps 2 to 4 on the other side of the wafer,
(6) etching the Si wafer to realize a V-groove and a flow sensing element cavity inside the wafer.
